(19) United States
(12) Patent Application Publication
(10) Pub. No.: US 2009/0325273 A1
(43) Pub. Date: Dec. 31, 2009

(54) MULTI-FUNCTIONAL TREATING DEVICE FOR TREATING PATHOLOGICAL TISSUE SECTIONS

(76) Inventors: CHIN PIN CHANG CHIEN, Kaohsiung City (TW); KANG TING LIU, Sijhih City (TW)

Correspondence Address: SINORICA, LLC, 2275 Research Blvd., Suite 500, ROCKVILLE, MD, 20850, US

(21) Appl. No.: 12/164,210

(22) Filed: Jun. 30, 2008

(51) Int. Cl. C12M 1/00

(52) U.S. Cl. 435/283.1

(57) ABSTRACT

A multi-functional treating device for pathological tissue sections includes a main body provided with at least one accommodating chamber formed in its top portion and a central controller. A water bath, an oven, a paraffin scrubber and a section baking rack are respectively installed in each of the accommodating chambers. The central controller is provided with plural control buttons, which are respectively connected with an electrothermal device installed under the paraffin scrubber, the water bath, the oven and the section baking rack respectively. Thus, the multi-functional treating device can simultaneously do jobs of scrubbing off extra paraffin, wetting tissue sections, primarily drying up tissue sections, and drying up slides carrying the sections, saving a lot of space.

### BACKGROUND OF THE INVENTION

1. Field of the Invention

This invention relates to a multi-functional treating device for pathological tissue sections, particularly to one able to simultaneously scrub off extra paraffin, wet and flat tissue sections, dry up tissue sections primarily, and get rid of extra moisture in tissue sections and extra paraffin carried by slides.

2. Description of the Prior Art

Commonly, a tissue section embedded by paraffin is a most predominant histology used to examine whether the histiocyte of animals or plants has pathological changes as it can be swiftly made to prevent it from perishing or deteriorating to lose its original structure. Because most of cell tissues are soft, it is very difficult to cut them into thin sections for a microscopic examination. Therefore, the tissues are in advance embedded with paraffin to keep them stiffened so as to be easily sliced. The procedures of treating the tissues are described below. First, the soft tissues are orderly dehydrated, embedded in a cassette with paraffin, stiffened, and cut into sections after scrubbing off the extra paraffin. Next, the sections are immersed in water to keep them flatly stretched out because the tissues may shrink to get curled after dehydrated. Then, the wetted flat sections are laid on slides and dried up to keep the paraffin transparent so as to be clearly examined under a microscope. However, the jobs to scrub extra paraffin off a section, to wet and stretch the section, to primarily dry up the section, and to further dry up the section laid on a slide are independently carried out by different rather huge devices, which occupy too much space.

### SUMMARY OF THE INVENTION

The object of this invention is to offer a multi-functional treating device able to simultaneously do jobs of scrubbing off extra paraffin, wetting a section to keep it flat, primarily drying up the section, and further drying up the section carried by a slide for pathological examination.

The main characteristics of the invention are a paraffin scrubber, a water bath, an oven and a section baking rack. A main body is provided with at least an accommodating chamber formed in its top portion for positioning related components, and a central controller with diverse functions. The paraffin scrubber is positioned in one of the accommodating chambers, employed to scrub off and recycle the extra paraffin formed around an embedding cassette carrying a tissue section. The water bath is installed in one of the accommodating chambers, used to wet tissue sections to keep them flatly extended so as to be well examined. The oven is fitted in one of the accommodating chambers for getting rid of water resting around the wetted tissue sections. The section baking rack is also installed in one of the accommodating chambers, employed to dry up extra water and paraffin in the tissue sections to make them transparent for being well examined.

The central controller is provided with plural control buttons respectively connected with an electrothermal device that is installed under the paraffin scrubber, the water bath, the oven and the section baking rack respectively and operated in accordance with a preset temperature.

Thus, the multi-functional treating device can simultaneously do jobs of scrubbing off extra paraffin, wetting the tissue sections, primarily drying up the tissue sections, and further drying up the tissue section carried by slides, saving a lot of space.

### BRIEF DESCRIPTION OF DRAWINGS

This invention is better understood by referring to the accompanying drawings, wherein:

FIG. 1 is a perspective view of a preferred embodiment of a multi-functional treating device for pathological tissue sections in the present invention;

FIG. 2 is an exploded perspective view of the preferred embodiment of a multi-functional treating device for pathological tissue sections in the present invention;

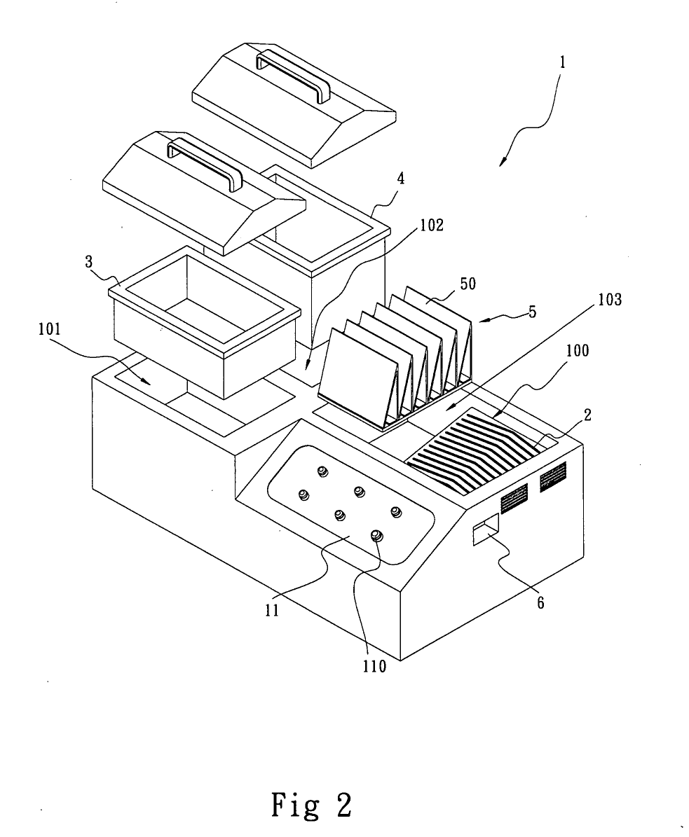

FIG. 3 is a top view of the preferred embodiment of a multi-functional treating device for pathological tissue sections in the present invention;

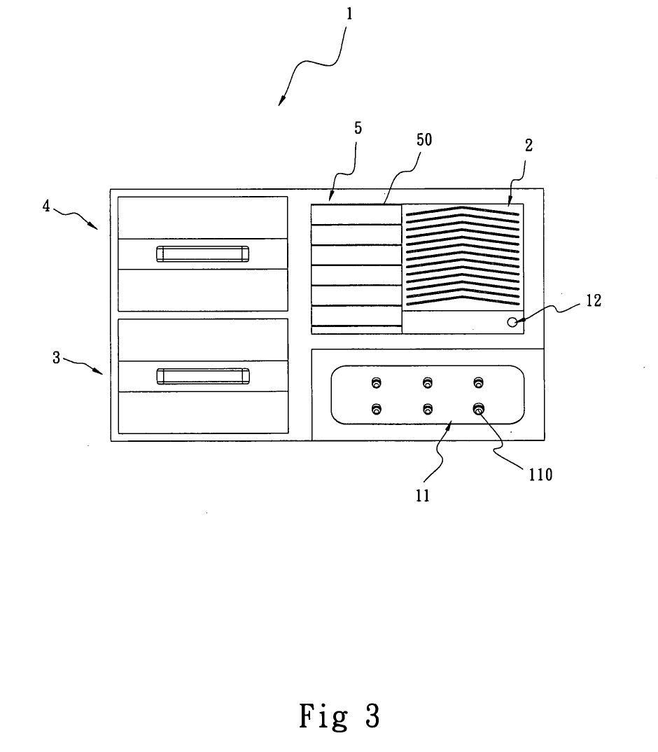

FIG. 4 is a flow chart of treating tissue sections in the present invention;

FIG. 5 is a cross-sectional view of an oven of the preferred embodiment of a multi-functional treating device for pathological tissue sections in the present invention;

FIG. 6 is a perspective view of a section baking rack of the preferred embodiment of a multi-functional treating device for pathological tissue sections in the present invention; and

FIG. 7 is a side view of the section baking rack of the preferred embodiment of a multi-functional treating device for pathological tissue sections in the present invention.

### DETAILED DESCRIPTION OF THE PREFERRED EMBODIMENT

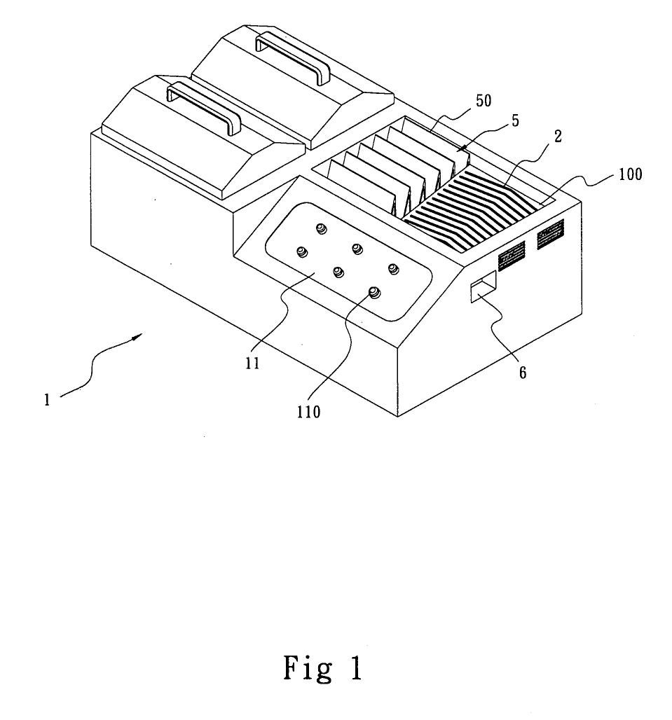

As shown in FIGS. 1˜4, a preferred embodiment of a multi-functional treating device for pathological tissue sections in the present invention includes a main body 1, a paraffin scrubber 2, a water bath 3, an oven 4 and a section baking rack 5.

The main body 1 is provided with four accommodating chambers 100, 101, 102 and 103 formed in its top portion for fitting related components therein, and a multi-functional central controller 11.

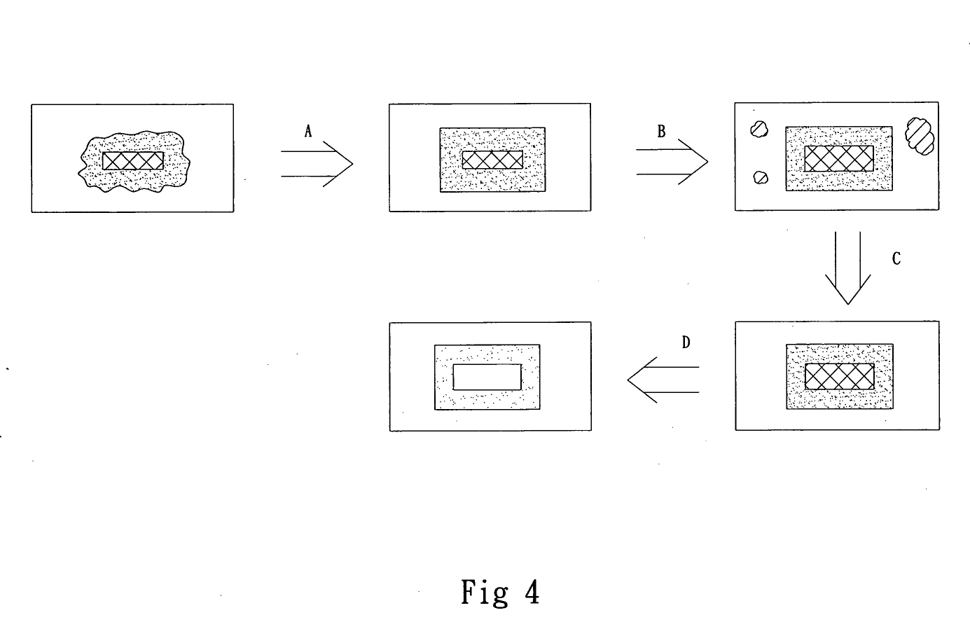

The paraffin scrubber 2 is positioned in the accommodating chamber 100, employed to scrub off and recycle the extra paraffin formed around an embedding cassette carrying a tissue section, as shown at the stage of (A) in FIG. 4.

The water bath 3 is installed in the accommodating chamber 101, used to wet tissue sections to keep them flatly extended so as to be well examined, as shown at the stage of (B) in FIG. 4. Moreover, the water bath 3 is easy to be positioned in and disassembled from the accommodating chamber 101.

The oven 4 is fitted in the accommodating chamber 102, used to position the wetted tissue sections in place while being dried up, as shown at the stage of (C) in FIG. 4. By the time, the sections are still embedded by paraffin to have a foggy appearance, needed to be further treated before examined.

The section baking rack 5 is installed in the accommodating chamber 103, provided with plural blocking plates 50 employed to put slides laid with the tissue sections, so that moisture in tissues and paraffin can be evaporated to enable the sections sufficiently transparent for being precisely examined, as shown at the stage of (D) in FIG. 4.

The central controller 11 is provided with plural control buttons 110 respectively connected with an electrothermal device 111 (not shown in Figures) that is properly installed under the paraffin scrubber 2, the water bath 3, the oven 4 and the section baking rack 5 respectively. The tissue sections can be appropriately baked with a temperature set in accordance to practical requirements. By means of the electro-thermal device 111, paraffin scrubbed off by the paraffin scrubber 2 can be melted to flow from a recycling vent 12 of the main body 1 into a recycling tank 6.

In order to lower the weight of the multi-functional treating device, the main components are preferably made of aluminum. With Teflon coated on the water bath 3, the oven 4 and the section baking rack 5, paraffin attached on the water bath 3, the oven 4 and the section baking rack 5 can be easily erased off. And, the paraffin scrubber 2, the water bath 3, the oven 4 and the section baking rack 5 can be timely taken out of the main body 1 when necessary.

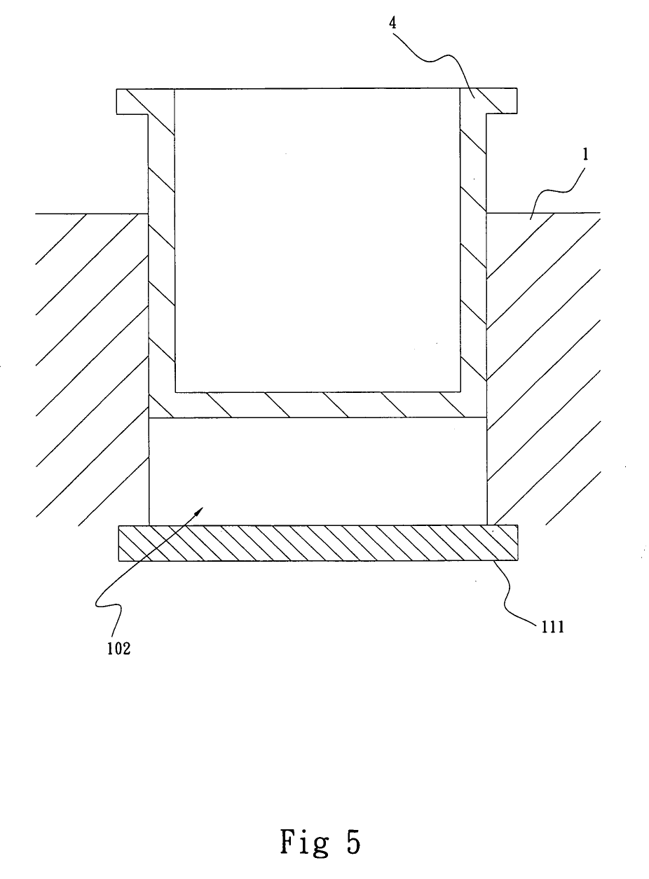

As mentioned previously, the main body 1, the paraffin scrubber 2, the water bath 3, the oven 4 and the section baking rack 5 are all made of metal with a good thermal conductivity. Taking the oven 4 showed in FIG. 5 as an example, the electrothermal device 111 is installed under the oven 4 to generate heat conducted from the main body 1 to the oven 4 positioned in the accommodating chamber 102, so that the tissue sections can be dried up in accordance with a temperature set in advance.

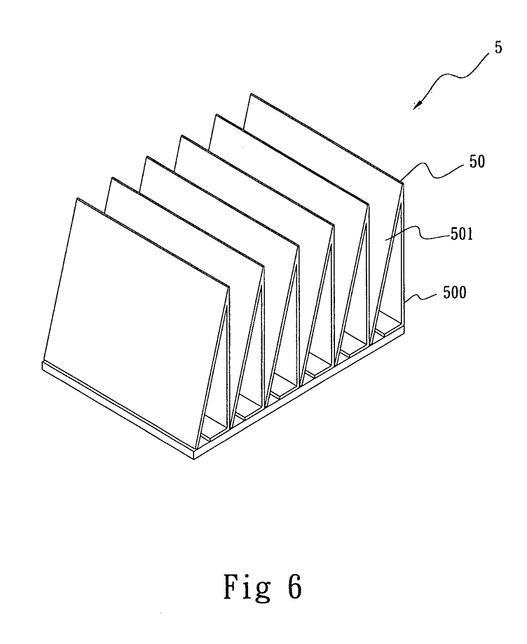

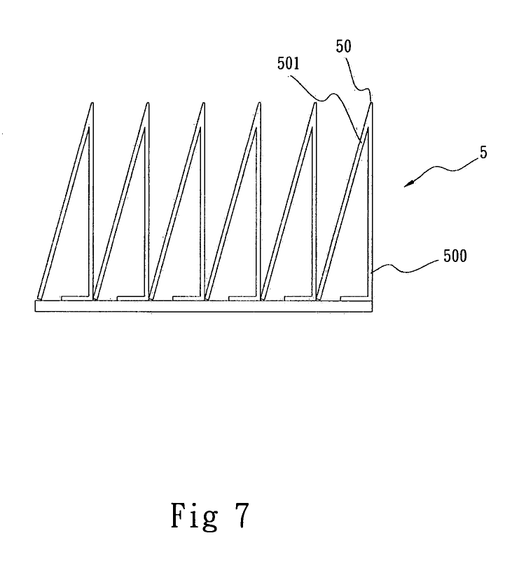

And, as shown in FIGS. 6 and 7, the blocking plates 50 of the section baking rack 5 are obliquely positioned and made of metal with a good thermal conductibility, respectively provided with a vertical portion 500 and a sloping portion 501 connected to each other. In using, a slide carrying a section is laid on the sloping portion 501. By means of the electrothermal device 111 (not shown in FIGS. 6 and 7), heat is conducted to the blocking plates 50, so that the section carried by the slide laid on the sloping portion 501 can be gradually dried up. As the section baking rack 5 is composed of a plurality of the blocking plates 50, it can be simultaneously deposited with at least one slide.

Thus, the invention has the following advantages as can be seen from the foresaid description.

1. With the paraffin scrubber 2, the water bath 3, the oven 4 and the section baking rack 5 installed together, the multi-functional treating device of the invention can not only save a lot of space, but also save time for a user who has to move about in case of operating several conventional independent devices.

2. The extra paraffin scrubbed off by the paraffin scrubber 2 can be reused for a reduction of cost.

3. The paraffin scrubber 2, the water bath 3, the oven 4 and the section baking rack 5 can be timely installed in or removed from the main body 1, enabling the multi-functional device conveniently operated.

While the preferred embodiment of the invention has been described above, it will be recognized and understood that various modifications may be made therein and the appended claims are intended to cover all such modifications that may fall within the spirit and scope of the invention.

What is claimed is:

1. A multi-functional treating device for pathological tissue sections, said multi-functional device comprising:
a main body provided with at least one accommodating chamber formed in its top portion for containing related component and a central controller provided with a plurality of control buttons that are respectively connected with an electrothermal device;
a water bath positioned in one of said accommodating chambers;
an oven installed in one of said accommodating chambers;
a paraffin scrubber fixed in one of said accommodating chambers; and
a section baking rack fitted in one of said accommodating chambers and provided with at least a blocking plate.

2. The multi-functional device treating for pathological tissue sections as claimed in claim 1, wherein said electrothermal device is installed under said paraffin scrubber, said water bath, said oven and said section baking rack respectively.

3. The multi-functional treating device for pathological tissue sections as claimed in claim 1, wherein said paraffin scrubber, said water bath, said oven and said section baking rack are made of metal.

4. The multi-functional treating device for pathological tissue sections as claimed in claim 3, wherein said metal is aluminum.

5. The multi-functional treating device for pathological tissue sections as claimed in claim 1, wherein said water bath has its surface coated with Teflon.

6. The multi-functional device treating for pathological tissue sections as claimed in claim 1, wherein said oven has its surface coated with Teflon.

7. The multi-functional treating device for pathological tissue sections as claimed in claim 1, wherein said section baking rack has its surface coated with Teflon.
